(19) United States
(12) Patent Application Publication
(10) Pub. No.: US 2026/0124074 A1
(43) Pub. Date: May 7, 2026

(54) WEARABLE HAPTIC FEEDBACK DEVICE

(71) Applicant: UNIVERSITY OF UTAH RESEARCH FOUNDATION, Salt Lake City, UT (US)

(72) Inventors: Jungkyu Kim, Salt Lake City, UT (US); Haohan Zhang, Salt Lake City, UT (US); Gonzalo Tello, Salt Lake City, UT (US); Kanghoon Choi, Salt Lake City, UT (US)

(21) Appl. No.: 19/117,946

(22) PCT Filed: Oct. 5, 2023
(86) PCT No.: PCT/US2023/076094

§ 371 Date: Apr. 2, 2025

Related U.S. Application Data
(60) Provisional application No. 63/413,690, filed on Oct. 6, 2022.

(51) Int. Cl. A61F 9/08; A61H 23/04

(52) U.S. Cl. CPC A61F 9/08; A61H 23/04; A61H 2201/165; A61H 2201/5071

(57) ABSTRACT

Examples provide a wearable device including a pump in fluid communication with a plurality of valves and a haptic output portion including a plurality of membranes respectively corresponding to the plurality of valves. The plurality of membranes are selectively adjustable between an active state and an inactive state in accordance with a state of the plurality of valves. The device also includes an electronic processor configured to receive input from an external device, encode the input to a haptic sequence, and provide haptic feedback to the user by generating a control signal to open and close selected ones of the plurality of valves according to the haptic sequence, wherein opening a valve causes a corresponding membrane to inflate.

### CROSS-REFERENCE TO RELATED APPLICATION(S)

This application claims priority to U.S. Provisional Ser. No. 63/413,690, filed on Oct. 6, 2022, the entire contents of which are fully incorporated herein by reference.

### FIELD

Embodiments herein relate to haptic feedback devices.

### SUMMARY

Visual impairments prevent many individuals from effectively reading information. Many individuals with visual impairments rely on audio description devices to consume text-based or other visual information. However, individuals with both hearing and visual impairments may rely primarily on tactile means for consuming information, such as Braille. Braille displays are typically expensive and large (e.g., tabletop devices), making them inaccessible for many individuals with visual impairments. Some vibratile feedback solutions exist, however, these options often lack portability. Additionally, studies indicate that users are more sensitive to normal forces than shear forces when receiving tactile feedback.

Thus, there is a need for a portable device for providing haptic feedback, such as Braille, to a user. One example provides a wearable device including a pump in fluid communication with a plurality of valves and a haptic output portion including a plurality of membranes respectively corresponding to the plurality of valves. The plurality of membranes are selectively adjustable between an active state and an inactive state in accordance with a state of the plurality of valves. The device also includes an electronic processor configured to receive input from an external device, encode the input to a haptic sequence, and provide haptic feedback to the user by generating a control signal to open and close selected ones of the plurality of valves according to the haptic sequence, wherein opening a valve causes a corresponding membrane to inflate.

In some aspects, the haptic output portion defines a contacting surface of the wearable device such that, when the wearable device is worn by the user, the haptic output portion contacts the skin of the user.

In some aspects, inflation of a membrane causes the membrane to output a normal force to the skin of the user.

In some aspects, the wearable device further includes a pressure sensor configured to measure a pressure in a fluid path between the pump and the plurality of valves, wherein the electronic processor is configured to detect a leak in the fluid path based on a signal received from the pressure sensor.

In some aspects, the electronic processor is further configured to, in response to detecting a leak in the fluid path, provide haptic feedback to the user indicating that a leak is detected.

In some aspects, the plurality of membranes includes six membranes arranged in a three by two array.

In some aspects, the haptic sequence is a Braille sequence.

In some aspects, each of the plurality of valves incudes an exhaust port, and the electronic processor is further configured to provide the haptic feedback to the user by, after opening a selected one of the plurality of valves, opening the exhaust port of the selected one of the plurality of valves.

In some aspects, each of the plurality of valves incudes a vacuum port, and the electronic processor is further configured to provide the haptic feedback to the user by, after opening a selected one of the plurality of valves, activating the vacuum port of the selected one of the plurality of valves.

In some aspects, the pump is configured to supply a constant pressure to the plurality of valves.

In some aspects, the constant pressure is 30 kilopascals.

In some aspects, the wearable device further includes a Darlington array configured to drive the plurality of valves based on the control signal.

In some aspects, the input includes at least one selected from the group consisting of text input, audio input, image input, and video input.

In some aspects, the electronic processor is communicatively connected to the external device by at least one selected from the group consisting of a universal serial bus (“USB”) connection, a Bluetooth connection, and a WiFi connection.

Another example provides a method implemented in a wearable device. The method includes receiving input from an external device, encoding the input to a haptic sequence, and providing haptic feedback to the user by generating a control signal to open and close selected ones of a plurality of valves according to the haptic sequence. A pump is in fluid communication with the plurality of valves, and a haptic output portion includes a plurality of membranes respectively corresponding to the plurality of valves. The plurality of membranes are selectively adjustable between and active state and an inactive state in accordance with a state of the plurality of valves. Opening a valve causes a corresponding membrane to inflate.

Other aspects of the invention will become apparent by consideration of the detailed description and accompanying drawings.

### BRIEF DESCRIPTION OF THE DRAWINGS

FIG. 1 illustrates a perspective view of a wearable haptic feedback device with portions of the housing removed, according to some examples.

FIG. 2 illustrates an enlarged view of a haptic output portion of a wearable device, according to some examples.

FIG. 3 illustrates a perspective view of a wearable haptic feedback device with portions of the housing removed, according to some examples.

FIG. 4 illustrates a block diagram of a wearable haptic feedback device, according to some examples.

FIG. 5 illustrates a block diagram of a haptic feedback controller, according to some examples.

FIG. 6 illustrates a method for providing haptic feedback to a user, according to some examples.

FIG. 7 illustrates a method for detecting a leak in a wearable haptic feedback device, according to some examples.

### DETAILED DESCRIPTION

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

Features illustrated or described as part of one embodiment can be used with another embodiment to yield a still further embodiment. Thus, it is intended that the present disclosure covers such modifications and variations as come within the scope of the appended claims and their equivalents. The detailed description uses numerical and letter designations to refer to features in the drawings. Like or similar designations in the drawings and description have been used to refer to like or similar parts of the invention.

As used herein, the terms “first”, “second”, and “third” may be used interchangeably to distinguish one component from another and are not intended to signify location or importance of the individual components. The singular forms “a,” “an,” and “the” include plural references unless the context clearly dictates otherwise. The terms “coupled,” “fixed,” “attached to,” and the like refer to both direct coupling, fixing, or attaching, as well as indirect coupling, fixing, or attaching through one or more intermediate components or features, unless otherwise specified herein. As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Terms of approximation, such as “generally,” “approximately,” or “substantially,” include values within ten percent greater or less than the stated value. When used in the context of an angle or direction, such terms include within ten degrees greater or less than the stated angle or direction. For example, “generally vertical” includes directions within ten degrees of vertical in any direction, e.g., clockwise or counter-clockwise.

Benefits, other advantages, and solutions to problems are described below with regard to specific embodiments. However, the benefits, advantages, solutions to problems, and any feature(s) that may cause any benefit, advantage, or solution to occur or become more pronounced are not to be construed as a critical, required, or essential feature of any or all the claims.

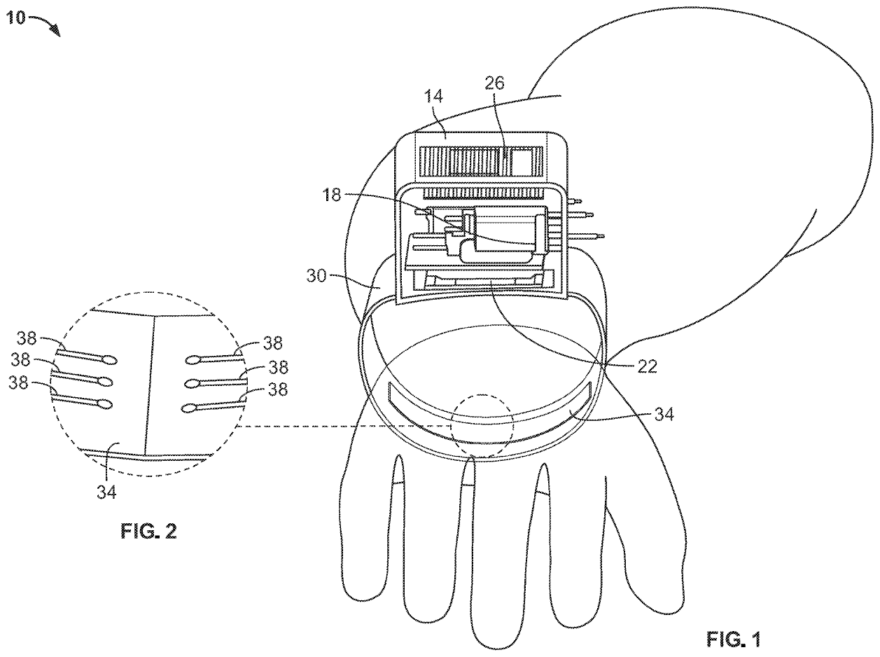

FIG. 1 illustrates a perspective view of a wearable, haptic feedback device 10, with portions of a housing 14 removed, according to some examples. The housing 14 supports an airflow control system 18, a tubing system 22 in fluid communication with the airflow control system 18, and a haptic feedback controller 26 electrically connected to the airflow control system 18. A flexible strap 30 may be connected to the housing 14 and enables a user to wear the wearable device 10 on, for example, a wrist, an arm, an ankle, or the like. The strap 30 may otherwise be referred to herein as a wristband 30. In some examples, the housing 14 has a volume of no more than approximately 132 cubic centimeters. For example, the housing 14 may have a width less than or equal to 60 millimeters (“mm”) (e.g., approximately 57 mm, approximately 50 mm, approximately 45 mm, etc.), a height less than or equal to 55 mm (e.g., approximately 51 mm, approximately 45 mm, approximately 40 mm, etc.), and a depth less than or equal to 40 mm (e.g., approximately 38 mm, approximately 35 mm, approximately 30 mm, etc.). The wearable device 10 may weigh less than, for example, 130 grams (e.g., approximately 120 grams, approximately 115 grams, approximately 100 grams, etc.).

The tubing system 22 is, for example, a microfluidic tubing system that extends through and interior of the wristband 30 to a haptic output portion 34 embedded in the wristband 30. The haptic output portion 34 may define a contacting surface of the wearable device 10 such that, when the wearable device 10 is worn by the user, the haptic output portion 34 contacts the skin of the user (e.g., the user's wrist) to provide hands-free haptic feedback. However, in some instances, the haptic output portion 34 is disposed on an exterior side of the wristband 30 opposite a contacting side of the wristband 30. The haptic output portion 34 may made be made of a flexible or rubber-like material. For example, the haptic output portion 34 may be made of a microfluidic silicone strip.

Referring now to FIG. 2, an enlarged view of a portion of the haptic output portion 34 is illustrated, according to some examples. The haptic output portion 34 includes a plurality of adjustable membranes 38 configured to selectively increase in volume (e.g., inflate) to provide a normal force to the user. In some instances, each membrane 38 has a thickness of approximately 100 micrometers. In some instances, each membrane 38 is made of a different material than a surrounding portion of the haptic output portion 34. In the illustrated example, the haptic output portion 34 includes six membranes 38 arranged in, for example, a three by two array. However, the haptic output portion 34 may include more than six membranes 38 or less than six membranes 38. Additionally, an arrangement of the membranes 38 may vary according to implementation. For example, in some instances, the haptic output portion 34 includes eighteen membranes 38 arranged in a three by six array, twenty-four membranes 38 arranged in a four by eight array, or the like. The haptic output portion 34 will be described in greater detail below with respect to FIG. 4.

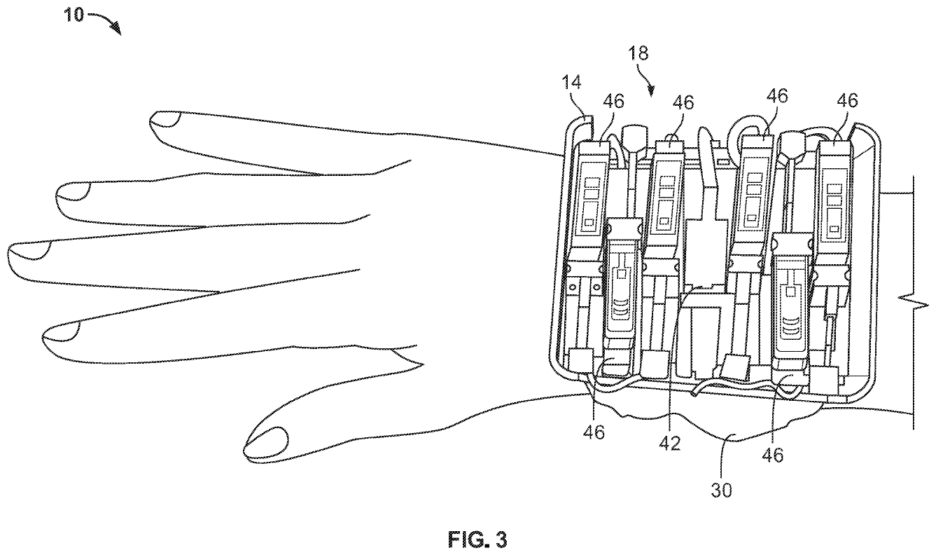

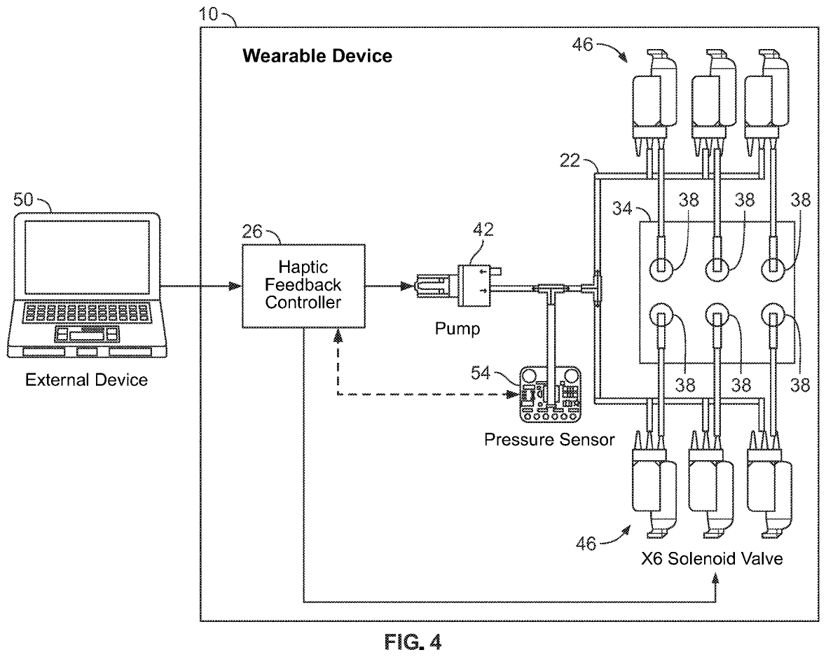

FIG. 3 illustrates another perspective view of the wearable device 10 with a top portion of the housing 14 removed, exposing the airflow control system 18, according to some examples. The airflow control system 18 includes pump 42 (e.g., a diaphragm pump) configured to provide pneumatic pressure to a plurality of valves 46 (e.g., solenoid microvalves). However, in some instances, the pump 42 provides hydraulic pressure to the plurality of valves 46. The pump 42 may be operable to pump air both into and out of the airflow control system 18 of the wearable device 10. The pump 42 may provide a constant supply of pressure to the airflow control system 18. For example, the pump 42 may provide a constant pressure of 25 kilopascals “kPa”), 30 kPa, 35 kPa, or the like.

Each of the plurality of valves 46 may correspond to a respective one of the plurality of membranes 38. Each of the plurality of valves 46 includes a pressure port configured to receive pneumatic pressure from the pump 42. Opening a pressure port of a respective valve 46 causes air received from the pump 42 to flow through the tubing system 22 and inflate a corresponding membrane 38 of the haptic output portion 34. When a user is wearing the wearable device 10, inflation of a corresponding membrane 38 causes the membrane 38 to provide a normal force to the skin of the user. Each of the plurality of valves 46 may further include a vacuum port and/or an exhaust port for releasing the pneumatic pressure, thus causing the corresponding membrane 38 to deflate.

FIG. 4 schematically illustrates a control system of the wearable device 10, according to some examples. The haptic feedback controller 26 is communicatively connectable to an external computing device 50 and configured to receive input from the external device 50. The external device 50 may be, for example, a mobile phone, a laptop computer, a tablet computer, a netbook computer, a personal computer (PC), a personal digital assistant (PDA), a desktop computer, or any programmable electronic device capable of transmitting data to the haptic feedback controller 26. The haptic feedback controller 26 may be communicatively connected to the external device 50 by a universal serial bus (“USB”) connection, a Bluetooth connection, a WiFi connection, and/or the like.

Based on the input received from the external device 50, the haptic feedback controller 26 is configured to generate a haptic sequence, and control the plurality of valves 46 according to the haptic sequence. For example, the input received from the external device 50 may include text input, and the haptic feedback controller 26 may be configured to generate a Braille sequence based on the contents of the text. The Braille sequence may be a letter-for-letter translation of the text input to Braille. However, in some instances, the Braille sequence includes one or more shorthand translations of the text input to Braille. For example, the haptic feedback controller 26 may be pre-programmed or dynamically programmed by a user with various shorthands.

The haptic sequence that may be generated by the haptic feedback controller 26 is not limited to Braille, and may reflect other forms of tactile feedback. For example, the haptic sequence may be used to simulate the sensation of human touch (e.g., in social interactions), map haptic cues to navigational directions, and/or to render interaction forces during teleoperation tasks. Additionally, the input received from the external device 50 is not limited to text, and may include audio data, image data, video data, and/or the like. For example, the haptic feedback controller 26 may generate a haptic sequence based on audio or visual content of video data for sensory simulation. In some instances, the haptic feedback controller 26 transcribes audio to text, and generates a haptic sequence (e.g., a Braille sequence) based on the transcribed audio. In some instances, the haptic feedback controller 26 generates a description of visual content (e.g., image or video data), and generates a haptic sequence according to the generated description.

The haptic feedback controller 26 selects one or more of the valves 46, and controls the selected ones of the valves 46 to open and/or close according to the haptic sequence. For example, the wearable device 10 may include a Darlington array configured to drive the plurality of valves 46 based on a control signal received from the haptic feedback controller 26. As described above, opening of a valve 46 causes a corresponding membrane 38 of the haptic output portion 34 to inflate and provide a haptic feedback to the user. The haptic feedback controller 26 may present each portion, or cell, of the haptic sequence to the user for a predetermined period of time (e.g., 0.5 seconds, one second, three seconds, etc.), or until a confirmation is received from the user (e.g., via a button, a touch screen, a vocal confirmation, or the like). In some instances, the predetermined period of time is a user-defined period of time. For example, the haptic feedback controller 26 may receive a selection by a user (e.g., via the external device 50, or via one or more buttons or dials included in the wearable device 10) of a preferred Braille reading speed (e.g., 100 words per minute (“WPM”), 120 WPM, 200 WPM, 300 WPM, etc.), wherein the predetermined period of time is based on the preferred reading speed.

After the predetermined period of time has expired or after receiving confirmation from the user to proceed to the next cell of the haptic sequence, the haptic feedback controller 26 controls the selected ones (e.g., the open ones) of the valves 46 to release pressure from the corresponding membranes 38 (e.g., by closing the pressure ports and opening the exhaust and/or vacuum ports of the selected valves 46). In some instances, when a next cell of the haptic sequence includes one or more of the same active membranes 38 (e.g., the same inflated membranes 38) as the current cell of the sequence, the haptic feedback controller 26 does not release the corresponding one or more same membranes 38, such that the one or more same membranes 38 remain active for the next cell of the sequence.

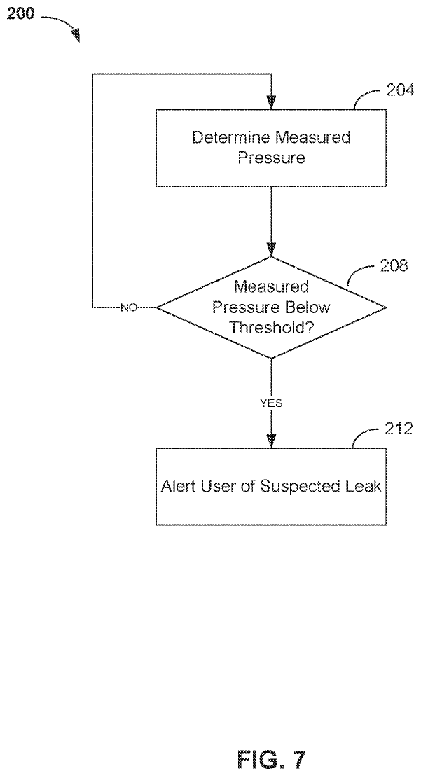

In some instances, the wearable device 10 further includes a pressure sensor 54 configured to measure a pressure in the tubing system 22 of the wearable device 10. For example, the pressure sensor 54 may be arranged to measure a pressure in the fluid path between the pump 42 and the plurality of valves 46. The pressure sensor 54 may output a signal to the haptic feedback controller 26 indicative of the measured pressure in the fluid path. Based on the signal received from the pressure sensor 54, the haptic feedback controller 26 may determine one or more system characteristics of the wearable device 10, and store the system characteristics in memory. The system characteristics may include, for example, an average pressure in the tubing system 22, pressure fluctuations in the tubing system 22, or information relating to suspected leaks in the tubing system 22, or information relating to suspected faults in the air pump 42. For example, the haptic feedback controller 26 may determine the presence of a leak in the tubing system 22 based on the measured pressure in the fluid path falling below a threshold (e.g., below 25 kPa, below 20 kPa, below 10 kPa, or the like). In response to detecting the leak, the haptic feedback controller 26 may generate a haptic sequence alerting the user of the leak, describe di greater detail below with respect to FIG. 7.

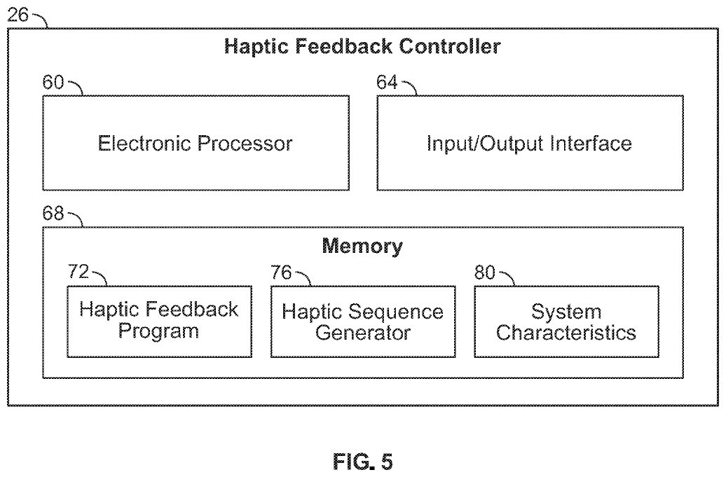

FIG. 5 illustrates a block diagram of the haptic feedback controller 26, according to some aspects. The haptic feedback controller 26 includes an electronic processor 60 (for example, a microprocessor, application specific integrated circuit, etc.), electrically connected to an input/output interface 64 and a memory 68.

The memory 68 may be made up of one or more non-transitory computer-readable media and includes at least a program storage area and a data storage area. The program storage area and the data storage area can include combinations of different types of memory, such as read-only memory (“ROM”), random access memory (“RAM”), electrically erasable programmable read-only memory (“EEPROM”), flash memory, or other suitable memory devices. The electronic processor 60 sends and receives information (for example, from the memory 68 and/or the input/output interface 64) and processes the information by executing one or more software instructions or modules, capable of being stored in the memory 68, or another non-transitory computer readable medium. The software can include firmware, one or more applications, program data, filters, rules, one or more program modules, and other executable instructions.

The electronic processor 60 is configured to retrieve from the memory 68 and execute, among other things, software for performing the methods as described herein. In the example illustrated, the memory 68 stores, among other things, a haptic feedback program 72, a haptic sequence generator 76 (e.g., a text-to-Braille program and/or other sensory feedback configurations), and system characteristics 80 (e.g., system pressure, operational data, etc.). The input/output interface 64 transmits and receives information from devices external to the haptic feedback controller 26 (for example, the external device 50, the pressure sensor 54, the valves 46, the pump 42, and/or the like). It should be understood that the haptic feedback controller 26 may include additional components than those illustrated in FIG. 5 and in various configurations. For example, in some examples, the haptic feedback controller 26 includes multiple electronic processors 60, multiple input/output interfaces 64, multiple memory modules 68, or a combination thereof.

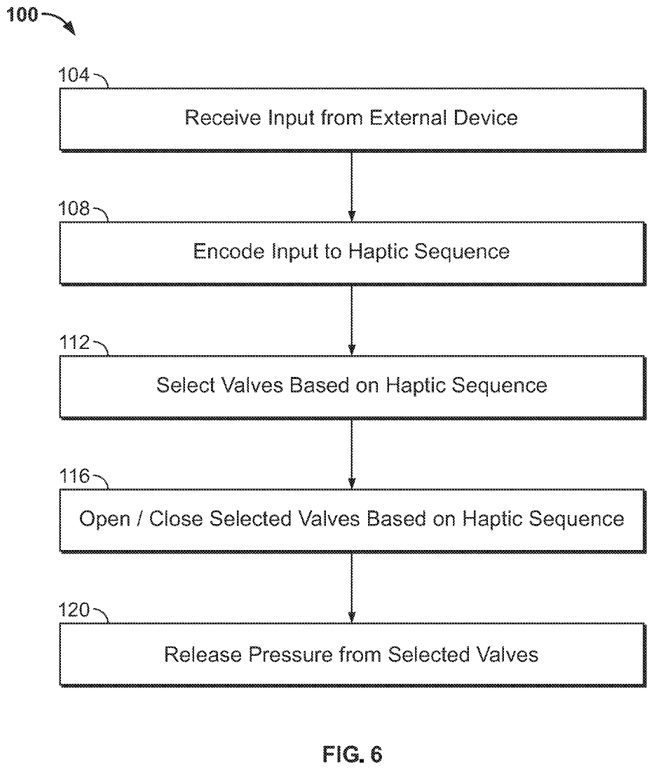

FIG. 6 illustrates an example method 100, implemented by the haptic feedback controller 26 (e.g., using the electronic processor 60), for providing haptic feedback to a user. The method 100 includes receiving input (e.g., text input, audio input, video input, image input, etc.) from the external device 50 (at block 104), and encoding the input to a haptic sequence, for example, using the haptic sequence generator 76 (at block 108). The method 100 further includes selecting one or more of the plurality of valves 46 based on the haptic sequence (at block 112), and generating a control signal to open and close the selected ones of the plurality of valves 46 according to the haptic sequence, causing the corresponding membranes 38 to inflate (at block 116). The method 100 may further include releasing pressure from the corresponding membranes 38 by opening the exhaust port and/or vacuum port of the selected valves 46 (at block 120).

Referring now to FIG. 7, an example method 200 implemented by, for example, the haptic feedback controller 26, for detecting a leak in the wearable device 10 is illustrated. The method 200 includes determining, based on a signal received form the pressure sensor 54, a measured pressure in the fluid path between the pump 42 and the plurality of valves 46 (at block 204). Based on the determined measured pressure, the haptic feedback controller 26 determines whether the measured pressure is below a threshold pressure indicative of a suspected leak in the wearable device (e.g., in the tubing system 22 of the wearable device) (at block 208). The threshold pressure may be a predetermined value or percentage less than the value of constant air pressure the pump 42 is configured to provide. For example, the threshold pressure may be 25 kPa, 20 kPa, 10 kPa, or the like.

In response to determining that the measured pressure is not less than the threshold pressure (“NO” at block 208), the haptic feedback controller 26 repeats the steps of the method 200. In contrast, in response to determining that the measured pressure is less than the threshold pressure (“YES” at block 208), the haptic feedback controller 26 alerts the user of a suspected leak in the wearable device 10 (at block 212). The haptic feedback controller 26 may alert the user by generating a haptic sequence indicative of the suspected leak.

In some instances, the leak may be severe enough that there is insufficient pressure in the tubing system 22 to inflate the membranes 38. In such instances, the haptic feedback controller 26 may alert the user of the suspected leak using an alternative means. For example, the haptic feedback controller 26 may alert the user of the suspected leak by transmitting an alert to another user device, such as the external device 50. In some instances, the wearable device 10 further includes a vibration motor, and the haptic feedback controller 26 activates the vibration motor in order to provide a vibratile alert to the user of the suspected leak. In some instances, the wearable device 10 further includes a visual indicator (e.g., an LED) and/or a speaker, and the haptic feedback controller 26 provide a visual alert and/or audible alert to the user in response to detecting a suspected leak. The haptic feedback controller 26 may compare the measured pressure to a second threshold pressure (e.g., a threshold pressure lower than the first threshold pressure) to determine whether there is sufficient pressure in the tubing system 22 to provide the alert by inflating the valves 46.

While the location of the suspected leak may be in the fluid path between the air pump 42 and the plurality of valves 46, the haptic feedback controller 26 may also detect suspected leaks in other portions of the tubing system 22. For example, the haptic feedback controller 26 may detect a leak in an air tube connecting a respective one of the plurality of valves 46 to a corresponding membrane 38 based on a change in measured pressure when the respective valve 46 is open. For example, when a leak is present in the tubing between the respective valve 46 and the corresponding membrane 38, the pressure measured by the pressure sensor 54 may be above the threshold pressure when the respective valve 46 is closed, and only drop below the threshold pressure when the respective valve 46 is open.

In the foregoing specification, specific examples have been described. However, one of ordinary skill in the art appreciates that various modifications and changes can be made without departing from the scope of the claimed subject matter. Accordingly, the specification and figures are to be regarded in an illustrative rather than a restrictive sense, and all such modifications are intended to be included within the scope of present teachings.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separately claimed subject matter.

What is claimed is:

1. A wearable device comprising:
a pump in fluid communication with a plurality of valves;
a haptic output portion including a plurality of membranes respectively corresponding to the plurality of valves, the plurality of membranes selectively adjustable between an active state and an inactive state in accordance with a state of the plurality of valves; and
an electronic processor configured to:
receive input from an external device,
encode the input to a haptic sequence, and
provide haptic feedback to the user by generating a control signal to open and close selected ones of the plurality of valves according to the haptic sequence, wherein opening a valve causes a corresponding membrane to inflate.

2. The wearable device of claim 1, wherein the haptic output portion defines a contacting surface of the wearable device such that, when the wearable device is worn by the user, the haptic output portion contacts the skin of the user.

3. The wearable device of claim 2, wherein inflation of a membrane causes the membrane to output a normal force to the skin of the user.

4. The wearable device of claim 1, further comprising a pressure sensor configured to measure a pressure in a fluid path between the pump and the plurality of valves, wherein the electronic processor is configured to detect a leak in the fluid path based on a signal received from the pressure sensor.

5. The wearable device of claim 1, wherein the electronic processor is further configured to:
in response to detecting a leak in the fluid path, provide feedback to the user indicating that a leak is detected.

6. The wearable device of claim 1, wherein the plurality of membranes includes six membranes arranged in a three by two array.

7. The wearable device of claim 6, wherein the haptic sequence is a Braille sequence.

8. The wearable device of claim 1, wherein each of the plurality of valves incudes an exhaust port, and the electronic processor is further configured to provide the haptic feedback to the user by, after opening a selected one of the plurality of valves, opening the exhaust port of the selected one of the plurality of valves.

9. The wearable device of claim 1, wherein each of the plurality of valves incudes a vacuum port, and the electronic processor is further configured to provide the haptic feedback to the user by, after opening a selected one of the plurality of valves, activating the vacuum port of the selected one of the plurality of valves.

10. The wearable device of claim 1, wherein the pump is configured to supply a constant pressure to the plurality of valves.

11. The wearable device of claim 10, wherein the constant pressure is 30 kilopascals.

12. The wearable device of claim 1, further comprising a Darlington array configured to drive the plurality of valves based on the control signal.

13. The wearable device of claim 1, wherein the input includes at least one selected from the group consisting of text input, audio input, image input, and video input.

14. The wearable device of claim 1, wherein the electronic processor is communicatively connected to the external device by at least one selected from the group consisting of a universal serial bus (“USB”) connection, a Bluetooth connection, and a WiFi connection.

15. A method implemented in a wearable device, the method comprising:
receiving input from an external device;
encoding the input to a haptic sequence; and
providing haptic feedback to the user by generating a control signal to open and close selected ones of a plurality of valves according to the haptic sequence,
wherein a pump is in fluid communication with the plurality of valves, a haptic output portion includes a plurality of membranes respectively corresponding to the plurality of valves, the plurality of membranes are selectively adjustable between an active state and an inactive state in accordance with a state of the plurality of valves, and opening a valve causes a corresponding membrane to inflate.

16. The method of claim 15, wherein the haptic output portion defines a contacting surface of the wearable device such that, when the wearable device is worn by the user, the haptic output portion contacts the skin of the user.

17. The method of claim 16, wherein inflation of a membrane causes the membrane to output a normal force to the skin of the user.

18. The method of claim 15, further comprising:
measuring, with a pressure sensor, a pressure in a fluid path between the pump and the plurality of valves; and
detecting a leak in the fluid path based on a signal received from the pressure sensor.

19. The method of claim 18, further comprising:
in response to detecting the leak in the fluid path, providing feedback to the user indicating that a leak is detected.

20. The method of claim 15, wherein the plurality of membranes includes six membranes arranged in a three by two array.
